(19) United States
(12) Patent Application Publication
(10) Pub. No.: US 2026/0364312 A1
(43) Pub. Date: Nov. 19, 2026

(54) MULTI-CURVE STEERABLE SHEATH DESIGNS FOR IMPROVED MANEUVERABILITY

(71) Applicant: Boston Scientific Scimed, Inc., Maple Grove, MN (US)

(72) Inventors: Charles Gibson, Malden, MA (US); Miles Montgomery, East Gwillimbury (CA); Matthew DiCicco, Toronto (CA)

(21) Appl. No.: 19/676,353

(22) Filed: May 13, 2026

Related U.S. Application Data
(60) Provisional application No. 63/805,123, filed on May 13, 2025.

(51) Int. Cl. A61M 25/01

(52) U.S. Cl. CPC A61M 25/0147; A61M 25/0136; A61M 2025/0161

(57) ABSTRACT

A steerable medical device includes an elongate body having a proximal portion including a proximal end and a distal portion including a distal end. A lumen extends from the proximal end to the distal end. A first steering member has a proximal end connected to a first steering mechanism and a distal end anchored at a first anchor point in the distal portion of the elongate body. A second steering member has a proximal end connected to a second steering mechanism and a distal end anchored at a second anchor point proximal of the first anchor point. Selective actuation of the first steering mechanism and the second steering mechanism allows for the formation of simple, compound, and multi-planar curves of the elongate body.

### CROSS REFERENCE TO RELATED APPLICATIONS

This application claims priority to U.S. Provisional Patent Application No. 63/805,123 entitled “MULTI-CURVE STEERABLE SHEATH DESIGNS FOR IMPROVED MANEUVERABILITY,” filed May 13, 2025, which is hereby incorporated by reference in its entirety.

### TECHNICAL FIELD

The present disclosure relates to systems and methods for steering medical devices. More specifically, the present disclosure relates to systems and methods for steering medical devices into simple, compound, and multi-planar shapes using multiple pull rings affixed at different axial locations and a plurality of steering members affixed to the pull rings.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths or introducer sheaths, dilators, and other medical tools, which can be referred to as catheters in this disclosure.

To position a medical device within the body at a target site, some type of navigation mechanism is used, such as a steering feature incorporated into a catheter, or a steering mechanism incorporated into a guide sheath for directing a medical device to the target site. Clinicians manually manipulate or operate the catheter or sheath using the steering feature. To facilitate the advancement of devices through a patient's vasculature, the concurrent application of torque at a proximal portion of the device and the ability to deflect the distal portion of the device in a selected direction permits the clinician to adjust the direction of advancement of the distal portion of the device and to selectively position the distal portion of the device during an electrophysiological procedure. In many procedures, several devices may need to be used to reach a desired location or perform various steps of a procedure. The present disclosure is directed to a device that allows for the creation of different curve combinations to facilitate therapy delivery for multiple procedures.

### SUMMARY

Example 1 is a steerable medical device. The device includes an elongate body having a proximal portion including a proximal end and a distal portion including a distal end. A first steering member has a proximal end connected to a first steering mechanism and a distal end anchored at a first anchor point in the distal portion of the elongate body. A second steering member has a proximal end connected to a second steering mechanism and a distal end anchored at a second anchor point proximal of the first anchor point. Selective actuation of the first steering mechanism and the second steering mechanism allows for the formation of simple, compound, and multi-planar curves of the elongate body.

Example 2 is the device of Example 1, further comprising a first pull ring at the first anchor point, wherein the first steering member is attached to the first pull ring.

Example 3 is the device of Example 2, further comprising a second pull ring at the second anchor point, wherein the second steering member is attached to the second pull ring.

Example 4 is the device of Example 3, further comprising a third steering member having a proximal end connected to a third steering mechanism and a distal end anchored at a third anchor point proximal of the second anchor point.

Example 5 is the device of Example 4, further comprising a third pull ring at the third anchor point, wherein the third steering member is attached to the third pull ring.

Example 6 is the device of any of Examples 1 to 5, wherein the first steering member comprises a first pair of steering wires.

Example 7 is the device of any of Examples 1 to 6, wherein the second steering member comprises a second pair of steering wires.

Example 8 is the device of any of Examples 1 to 5, wherein the first steering member and the second steering member extend along a common radial location on the elongate body.

Example 9 is the device of any of Examples 1 to 7, wherein the first steering member extends along a first radial location on the elongate body and the second steering member extends along a second radial location on the elongate body.

Example 10 is the device of Example 9, wherein the first radial location is offset from the second radial location by 90 degrees.

Example 11 is the device of Example 9, wherein the first radial location is offset from the second radial location by 10-80 degrees.

Example 12 is the device of any of Examples 3 to 11, wherein at least one of the second pull ring and the third pull ring is configured for routing the first steering member underneath.

Example 13 is the device of any of Examples 3 to 12, wherein at least one of the second pull ring and the third pull ring has a non-circular cross-section.

Example 14 is the device of any of Examples 3 to 12, wherein at least one of the second pull ring and the third pull ring includes a channel for routing the first steering member.

Example 15 is the device of any of Examples 1 to 14, wherein the elongate body has portions of different stiffness.

Example 16 is a steerable medical device. The device includes an elongate body having a proximal portion including a proximal end and a distal portion including a distal end. A lumen extends from the proximal end to the distal end. A first steering member has a proximal end connected to a first steering mechanism and a distal end anchored at a first anchor point in the distal portion of the elongate body. A second steering member has a proximal end connected to a second steering mechanism and a distal end anchored at a second anchor point proximal of the first anchor point. Selective actuation of the first steering mechanism and the second steering mechanism allows for the formation of simple, compound, and multi-planar curves of the elongate body.

Example 17 is the device of Example 16, further comprising a first pull ring at the first anchor point, wherein the first steering member is attached to the first pull ring.

Example 18 is the device of Example 17, further comprising a second pull ring at the second anchor point, wherein the second steering member is attached to the second pull ring.

Example 19 is the device of Example 18, further comprising a third steering member having a proximal end connected to a third steering mechanism and a distal end anchored at a third anchor point proximal of the second anchor point.

Example 20 is the device of Example 19, further comprising a third pull ring at the third anchor point, wherein the third steering member is attached to the third pull ring.

Example 21 is the device of Example 16, wherein the first steering member comprises a first pair of steering wires.

Example 22 is the device of Example 16, wherein the second steering member comprises a second pair of steering wires.

Example 23 is the device of Example 16, wherein the first steering member and the second steering member extend along a common radial location on the elongate body.

Example 24 is the device of Example 16, wherein the first steering member extends along a first radial location on the elongate body and the second steering member extends along a second radial location on the elongate body.

Example 25 is the device of Example 24, wherein the first radial location is offset from the second radial location by 90 degrees.

Example 26 is the device of Example 24, wherein the first radial location is offset from the second radial location by 10-80 degrees.

Example 27 is the device of Example 20, wherein at least one of the second pull ring and the third pull ring is configured for routing the first steering member underneath.

Example 28 is the device of Example 20, wherein at least one of the second pull ring and the third pull ring has a non-circular cross-section.

Example 29 is the device of Example 20, wherein at least one of the second pull ring and the third pull ring includes a channel for routing the first steering member.

Example 30 is the device of Example 16, wherein the elongate body has portions of different stiffness.

Example 31 is a steerable medical device. The device includes an elongate body having a proximal portion including a proximal end and a distal portion including a distal end. A first steering member has a proximal end connected to a first steering mechanism and a distal end anchored to a first pull ring at a first anchor point in the distal portion of the elongate body. A second steering member has a proximal end connected to a second steering mechanism and a distal end anchored to a second pull ring at a second anchor point proximal of the first anchor point. The second pull ring is configured for routing the first steering member underneath. Selective actuation of the first steering mechanism and the second steering mechanism allows for the formation of simple, compound, and multi-planar curves of the elongate body.

Example 32 is the device of Example 31, wherein the second pull ring has a non-circular cross-section.

Example 33 is the device of Example 31, wherein the second pull ring includes a channel for routing the first steering member.

Example 34 is a steerable medical device. The device includes an elongate body having a proximal portion including a proximal end, a distal portion including a distal end, and an intermediate portion extending between the proximal portion and the distal portion. The distal portion has a first stiffness, the intermediate portion has a second stiffness, and the proximal portion has a third stiffness, the first stiffness being less than the second stiffness and the third stiffness. A first steering member has a proximal end connected to a first steering mechanism and a distal end anchored to a first pull ring at a first anchor point in the distal portion of the elongate body. A second steering member has a proximal end connected to a second steering mechanism and a distal end anchored to a second pull ring at a second anchor point proximal of the first anchor point. Selective actuation of the first steering mechanism and the second steering mechanism allows for the formation of simple, compound, and multi-planar curves of the elongate body.

Example 35 is the device of Example 34, wherein the distal portion extends from the distal end to the second anchor point.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a steerable medical device, in accordance with embodiments of the disclosure.

FIG. 2A a partial perspective view of a steerable medical device illustrating an arrangement of steering members and pull rings, in accordance with embodiments of the disclosure.

FIG. 2B is a side view of the steerable medical device of FIG. 2A, in accordance with embodiments of the disclosure.

FIGS. 3A and 3B illustrate various shapes of the steerable medical device achieved by the arrangement of FIG. 2A, in accordance with embodiments of the disclosure.

FIG. 4A is a cross-sectional view along line 4-4 of FIG. 2A illustrating an embodiment of a pull ring, in accordance with embodiments of the disclosure.

FIG. 4B is a cross-sectional view along line 4-4 of FIG. 2A illustrating an embodiment of a pull ring, in accordance with embodiments of the disclosure.

FIG. 4C is a cross-sectional view along line 4-4 of FIG. 2A illustrating an embodiment of a pull ring, in accordance with embodiments of the disclosure.

FIG. 5A a partial perspective view of a steerable medical device illustrating an arrangement of steering members and pull rings, in accordance with embodiments of the disclosure.

FIG. 5B is a side view of the steerable medical device of FIG. 5A, in accordance with embodiments of the disclosure.

FIG. 6 illustrates a shape of the steerable medical device achieved by the arrangement of FIG. 5A, in accordance with embodiments of the disclosure.

FIG. 7A is a cross-sectional view along line 7-7 of FIG. 5A illustrating an embodiment of a pull ring, in accordance with embodiments of the disclosure.

FIG. 7B is a cross-sectional view along line 7-7 of FIG. 5A illustrating an embodiment of a pull ring, in accordance with embodiments of the disclosure.

FIG. 7C is a cross-sectional view along line 7-7 of FIG. 5A illustrating an embodiment of a pull ring, in accordance with embodiments of the disclosure.

FIG. 7D is a cross-sectional view along line 7-7 of FIG. 5A illustrating an embodiment of a pull ring, in accordance with embodiments of the disclosure.

FIG. 7E is a cross-sectional view along line 7-7 of FIG. 5A illustrating an embodiment of a pull ring, in accordance with embodiments of the disclosure.

FIG. 7F is a cross-sectional view along line 7-7 of FIG. 5A illustrating an embodiment of a pull ring, in accordance with embodiments of the disclosure.

FIG. 8 is a perspective view of a steerable medical device, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

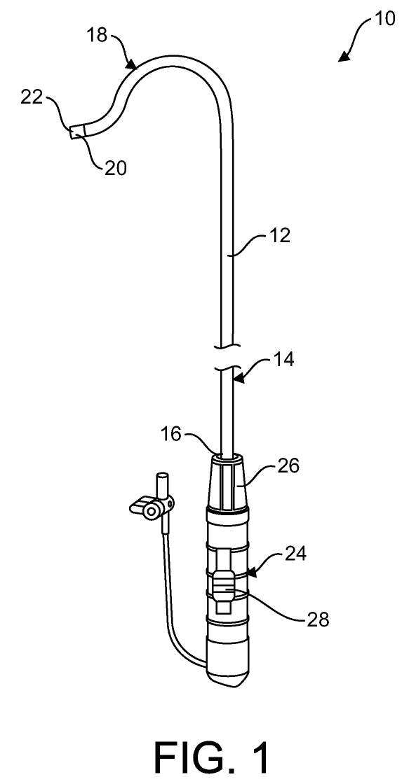

FIG. 1 is a perspective view of a steerable medical device 10, in accordance with embodiments of the disclosure. The steerable medical device 10 can take the form of a sheath, catheter, dilator, lead, guidewire, or other flexible, deflectable medical tool that can be steered to a desired target site within a patient. As illustrated in FIG. 1, the device 10 includes an elongate body 12 having a proximal portion 14 including a proximal end 16 and a distal portion 18 including a distal end 20. A lumen 22 extends from the proximal end 16 to the distal end 18. In some embodiments, the elongate body 12 is formed of several layers of materials. For example, in some embodiments, the elongate body 12 includes a liner surrounded by a support member such as a braid, coil, or slotted tube (e.g., a laser cut hypotube), and a polymeric material encasing the liner and the support member. The proximal end 16 of the elongate body 12 is connected to a handle 24. The handle 24 is configured for steering and manipulating the elongate body 12 to a desired location within a patient.

The distal portion 18 of the elongate body 12 is configured for creating simple, compound, and multi-planar shapes. This is achievable by the inclusion of multiple pull rings affixed at different axial locations along the elongate body 12 and a plurality of steering members affixed to the pull rings as discussed below. The steering members are affixed to the pull rings using adhesives, welding, mechanical techniques such as crimping, or by any other method suitable for permanently connecting the steering members and the pull rings.

The handle 24 includes at least one steering mechanism for applying tension to the steering members. In FIG. 1, the steerable medical device 10 includes a first steering mechanism 26 and a second steering mechanism 28. In various embodiments, the first steering mechanism 26 includes a rotatable knob that is rotatable in a first direction and a second direction. Rotation of the first steering mechanism 26 in a first direction, for example clockwise, causes tensioning of a first steering member and deflection of the distal portion 18 in a first deflection direction. Rotation of the first steering mechanism 26 in a second direction, for example counterclockwise, causes tensioning of a second steering member and deflection of the distal portion 18 in a second deflection direction. The second steering mechanism 28 includes a sliding actuator that can be manipulated by a user to apply selective tensioning to additional steering members for further deflection of the distal portion 18. In some embodiments, the first steering mechanism 26 and the second steering mechanism 28 both take the form of a rotatable knob. The rotational knob can be rotatable coaxially with the elongate body 12 as illustrated in FIG. 1 or can be configured to rotate perpendicularly to the longitudinal axis of the elongate body 12. In other embodiments, the first steering mechanism 26 and the second steering mechanism 28 both take the form of a sliding actuator. In various embodiments, one or more of the steering mechanisms is a paddle that rotates perpendicular to the sheath axis.

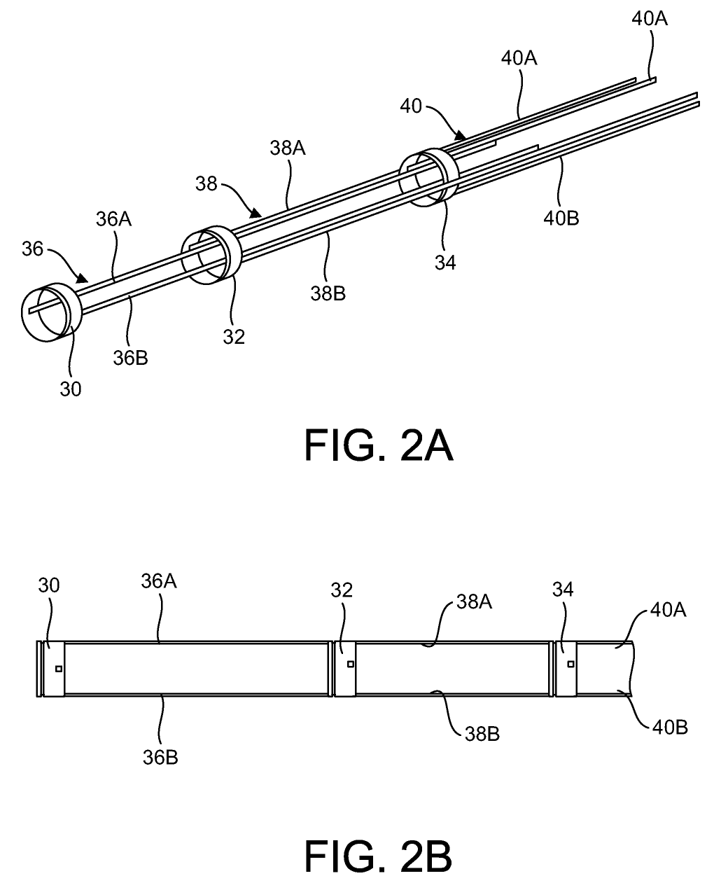

FIG. 2A a partial perspective view of a steerable medical device 10 illustrating an arrangement of steering members and pull rings, in accordance with embodiments of the disclosure. FIG. 2B is a side view of the steerable medical device of FIG. 2A. FIGS. 2A and 2B illustrate steering components in a distal portion 18 of the elongate body 12. Other elements forming the elongate body 12, such as a support member or polymeric material, are not shown in FIGS. 2A and 2B. The steering components of the medical device include a first pull ring 30, a second pull ring 32, and an optional third pull ring 34, a first steering member 36, a second steering member 38, and an optional third steering member 40. In some embodiments, the first pull ring 30 is located at the distal end 20 of the elongate body 12. In other embodiments, the first pull ring 30 is located proximal of the distal end 20 such that a tip of polymeric material extends distally beyond the first pull ring 30. The second pull ring 32 is located proximal of the first pull ring 30, and the third pull ring is located proximal of the second pull ring 32. While three pull rings 30, 32, 34 are illustrated in FIG. 2A, some embodiments include less than three. In an embodiment, including three pull rings, the handle 24 would further include a third steering mechanism (not shown). Such a mechanism could include, for example, a second radially rotating knob.

The first steering member 36 includes at least one steering wire having a distal end attached to a first anchor point on the first pull ring 30 and a proximal end connected with the first steering mechanism 26. As illustrated in FIG. 2A, the first steering member 36 includes a first pair of steering wires 36A, 36B. The first pair of steering wires 36A, 36B are arranged on opposite sides of the elongate body 12 such that tension applied to one of the pair of wires 36A, 36B causes deflection of the elongate body 12 in a first deflection direction and tension applied to the other of the pair of wires 36A, 36B causes deflection of the elongate body 12 in a second deflection direction that is opposite the first direction. In some embodiments, the first pair of steering wires 36A, 36B are arranged at different axial locations, but not opposite one another, such that the first deflection direction and the second deflection direction are different but not opposite.

The second steering member 38 includes at least one steering wire having a distal end attached to a second anchor point on the second pull ring 32 and a proximal end connected with the second steering mechanism 28. As illustrated in FIG. 2A, the second steering member 38 includes a second pair of steering wires 38A, 38B. The second pair of steering wires 38A, 38B are arranged on opposite sides of the elongate body 12 such that tension applied to one of the pair of wires 38A, 38B causes deflection of the elongate body 12 in a first deflection direction and tension applied to the other of the pair of wires 38A, 38B causes deflection of the elongate body 12 in a second deflection direction that is opposite the first direction. In some embodiments, the second pair of steering wires 38A, 38B are arranged at different axial locations, but not opposite one another, such that the first deflection direction and the second deflection direction are different but not opposite.

The optional third steering member 40 includes at least one steering wire having a distal end attached to a third anchor point on the third pull ring 34 and a proximal end connected to a third steering mechanism (not shown). In some embodiments, the third steering mechanism is incorporated into the first steering mechanism 26 or the second steering mechanism 28. As illustrated in FIG. 2A, the third steering member 40 includes a third pair of steering wires 40A, 40B. The third pair of steering wires 40A, 40B are arranged on opposite sides of the elongate body 12 such that tension applied to one of the pair of wires 40A, 40B causes deflection of the elongate body 12 in a first deflection direction and tension applied to the other of the pair of wires 40A, 40B causes deflection of the elongate body 12 in a second deflection direction that is opposite the first direction. In some embodiments, the third pair of steering wires 40A, 40B are arranged at different axial locations, but not opposite one another, such that the first deflection direction and the second deflection direction are different but not opposite.

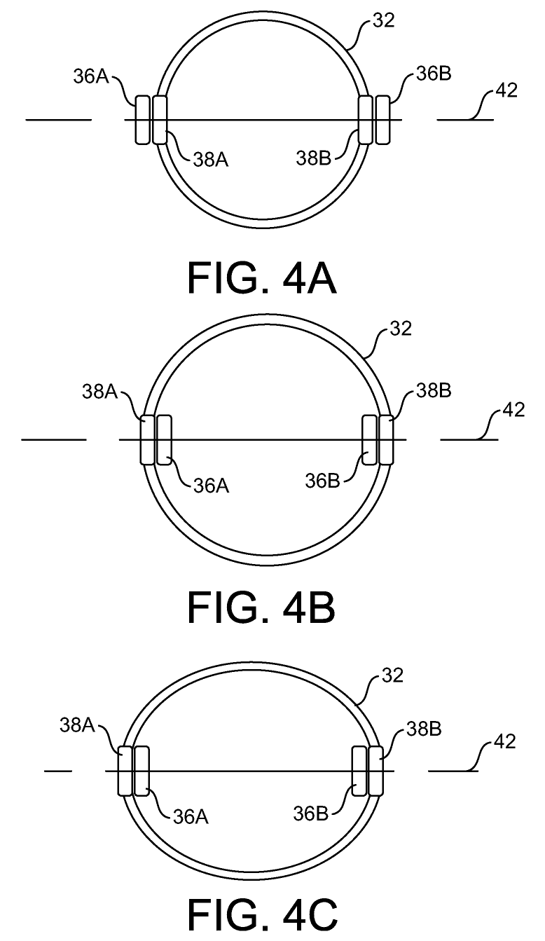

The first pair of steering wires 36A, 36B, the second pair of steering wires 38A, 38B, and the optional third pair of steering wires 40A, 40B extend along common radial locations on the elongate body 12. That is, first pair of steering wires 36A, 36B, the second pair of steering wires 38A, 38B, and the third pair of steering wires 40A, 40B reside in a common plane 42 (FIGS. 4A-4C) that transverses the elongate body 12.

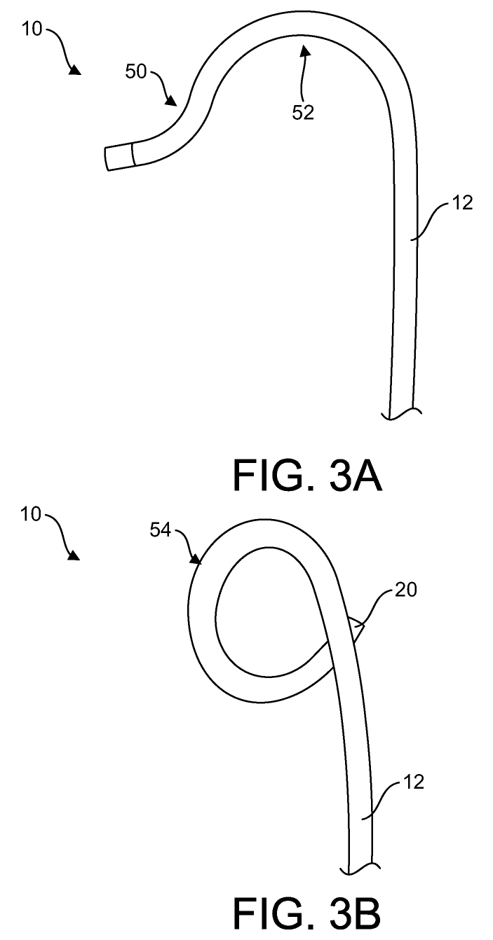

FIGS. 3A and 3B illustrate various shapes of the steerable medical device achieved by manipulating the first steering mechanism 26 and the second steering mechanism 28. The various shapes of the steerable medical device of FIGS. 3A and 3B remain substantially in the same plane because the first pair of steering wires 36A, 36B, the second pair of steering wires 38A, 38B, and the optional third pair of steering wires 40A, 40B reside in a common plane 42 (FIGS. 4A-4C).

In FIG. 3A, tension is applied to steering wire 36A of the first steering member 36 using the first steering mechanism 26. This causes the elongate body 12 to create a first bend 50 in the distal portion 18. Tension is also applied to steering wire 38B of the second steering member 28 using the second steering mechanism 28. This results in the formation of second bend 52 in the distal portion 18. Because steering wire 36A of the first steering member 36 is anchored to and resides on an opposite side of elongate body 12 from steering wire 38B of the second steering member 28, the first bend 50 curves in a direction that is opposite that of second bend 52, creating the S-shape as illustrated. While not illustrated, applying tension to steering wire 40A of an optional third steering member 40 would create a third bend in the same direction of first bend 50. In such an arrangement, the third bend would be proximal of the second bend 52 creating a serpentine shape of the distal portion 18. Applying tension to steering wire 40B of an optional third steering member 40 would result in second bend 52 being compounded and increased. A mirror image of the S-shape of FIG. 3A is created by tensioning steering wire 36B of the first steering member 36 and steering wire 38A of the second steering member 28.

In FIG. 3B, tension is applied to steering wire 36B of the first steering member 36 using the first steering mechanism 26 and to steering wire 38B of the second steering member 28 using the second steering mechanism 28. This results in the formation of single curve 54 in the distal portion 18 having a curvature that places the distal end 20 adjacent the elongate body 12. A mirror image of the shape of FIG. 3B is created by tensioning steering wire 36A of the first steering member 36 and steering wire 38A of the second steering member 28.

FIG. 4A is a cross-sectional view along line 4-4 of FIG. 2A illustrating an embodiment of a pull ring, in accordance with embodiments of the disclosure. As illustrated in FIG. 4A, the second pair of steering wires 38A, 38B of the second steering member 38 are integral with or affixed to the second pull ring 32. The first pair of steering wires 36A, 36B of the first steering member 36 are located on a common plane 42 as the second pair of steering wires 38A, 38B. The first pair of steering wires 36A, 36B stay outside of the second pair of steering wires 38A, 38B and remain on an exterior of the second pull ring 32.

FIG. 4B is a cross-sectional view along line 4-4 of FIG. 2A illustrating an embodiment of a pull ring, in accordance with embodiments of the disclosure. As illustrated in FIG. 4B, the second pull ring 32 is configured for routing the first pair of steering wires 36A, 36B of the first steering member 36 underneath. As illustrated in FIG. 4B, the second pair of steering wires 38A, 38B of the second steering member 38 are integral with or affixed to the second pull ring 32. The first pair of steering wires 36A, 36B of the first steering member share a common plane 42 with the second pair of steering wires 38A, 38B. The second pair of steering wires 38A, 38B are positioned on top of the first pair of steering wires 36A, 36B along the elongate body 12. As such, the first pair of steering wires 36A, 36B pass through the interior of the second pull ring 32 before reaching the first pull ring 30. In some embodiments, the third pull ring 34 is configured for routing both the first pair of steering wires 36A, 36B of the first steering member 36 and the second pair of steering wires 38A 38B of the second steering member 38 underneath.

FIG. 4C is a cross-sectional view along line 4-4 of FIG. 2A illustrating an embodiment of a pull ring, in accordance with embodiments of the disclosure. As illustrated in FIG. 4C, the second pull ring 32 has a non-circular cross-section for routing the first pair of steering wires 36A, 36B of the first steering member 36 underneath. The second pull ring 32 has an oval or elliptical cross-section. As illustrated in FIG. 4C, the second pair of steering wires 38A, 38B of the second steering member 38 are integral with or affixed to the second pull ring 32. The first pair of steering wires 36A, 36B of the first steering member share a common plane 42 with the second pair of steering wires 38A, 38B. The second pair of steering wires 38A, 38B are positioned on top of the first pair of steering wires 36A, 36B along the elongate body 12. As such, the first pair of steering wires 36A, 36B pass through the interior of the second pull ring 32 before reaching the first pull ring 30. In some embodiments, the third pull ring 34 is has a non-circular cross-section for routing both the first pair of steering wires 36A, 36B of the first steering member 36 and the second pair of steering wires 38A 38B of the second steering member 38 underneath.

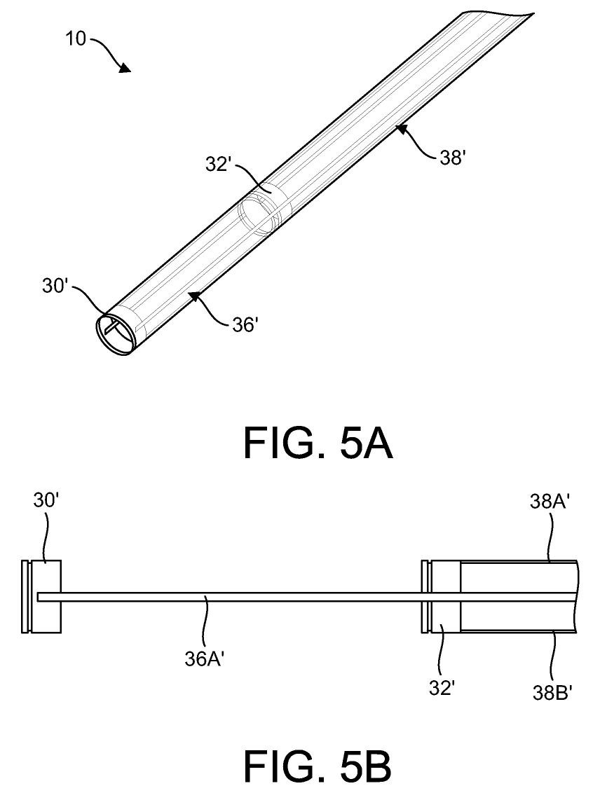

FIG. 5A a partial perspective view of a steerable medical device illustrating an arrangement of steering members and pull rings for creating multi-planar shapes, in accordance with embodiments of the disclosure. FIG. 5B is a side view of the steerable medical device of FIG. 5A. FIGS. 5A and 5B illustrate steering components in a distal portion 18 of the elongate body 12. Other elements forming the elongate body 12, such as a support member or polymeric material, are not shown in FIGS. 5A and 5B. The steering components of the medical device include a first pull ring 30’, a second pull ring 32’, a first steering member 36’, and a second steering member 38’. In some embodiments, the first pull ring 30’ is located at the distal end 20 of the elongate body 12. In other embodiments, the first pull ring 30’ is located proximal of the distal end 20 such that a tip of polymeric material extends distally beyond the first pull ring 30’. The second pull ring 32’ is located proximal of the first pull ring 30’. While two pull rings 30’, 32’ are illustrated in FIGS. 5A and 5B, some embodiments include more or less than two.

The first steering member 36’ includes at least one steering wire having a distal end attached to a first anchor point on the first pull ring 30’ and a proximal end connected with the first steering mechanism 26. As illustrated in FIG. 5A, the first steering member 36’ includes a first pair of steering wires 36A’, 36B’. The first pair of steering wires 36A’, 36B’ are arranged on opposite sides of the elongate body 12 such that tension applied to one of the pair of wires 36A’, 36B’ causes deflection of the elongate body 12 in a first deflection direction and tension applied to the other of the pair of wires 36A’, 36B’ causes deflection of the elongate body 12 in a second deflection direction.

The second steering member 38’ includes at least one steering wire having a distal end attached to a second anchor point on the second pull ring 32’ and a proximal end connected with the second steering mechanism 28. As illustrated in FIG. 5A, the second steering member 38’ includes a second pair of steering wires 38A’, 38B’. The second pair of steering wires 38A’, 38B’ are arranged on opposite sides of the elongate body 12 offset from the first pair of steering wires 36A’, 36B’ such that tension applied to one of the pair of wires 38A’, 38B’ causes deflection of the elongate body 12 in a third deflection direction and tension applied to the other of the pair of wires 38A’, 38B’ causes deflection of the elongate body 12 in a forth deflection direction.

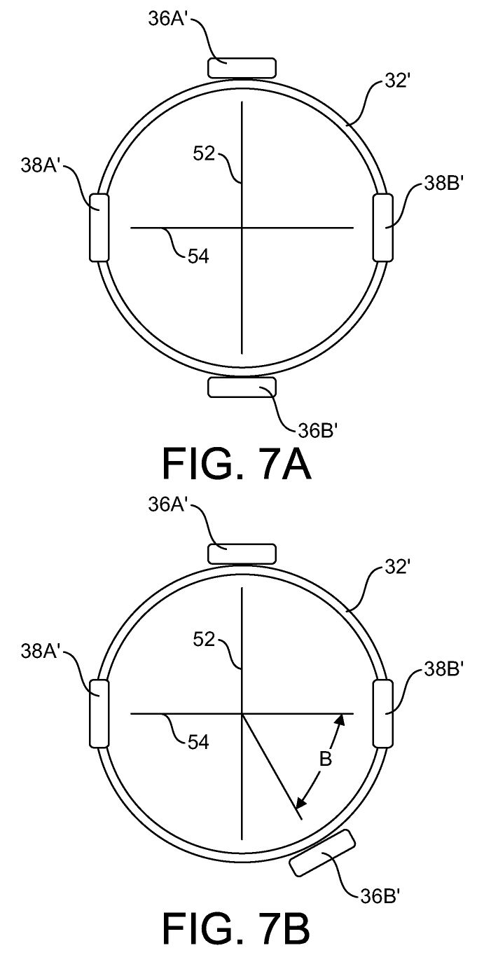

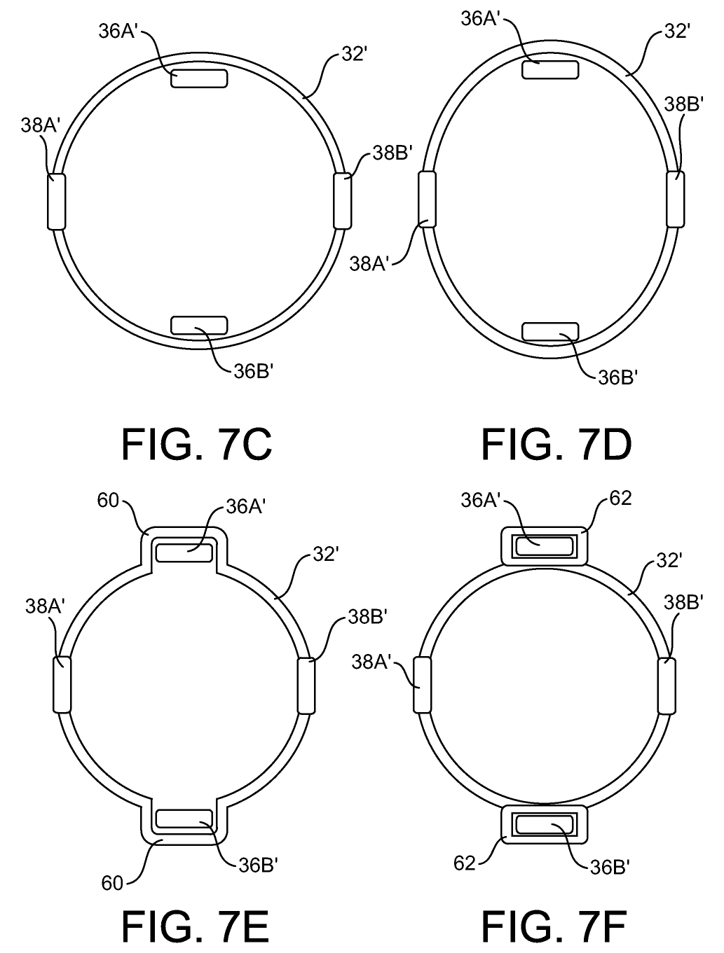

In the embodiment of FIGS. 5A and 5B, the first pair of steering wires 36A’, 36B’ of the first steering member 36’ extend along a first radial location on the elongate body 12 and the second pair of steering wires 38A’, 38B’ of the second steering member 38’ extend along a second radial location on the elongate body 12. In FIGS. 5A and 5B, the first radial location is offset from the second radial location by 90 degrees. This can be seen clearly in FIG. 7A. In FIG. 7A, the first pair of steering wires 36A’, 36B’ of the first steering member 36’ are arranged at radial locations on opposite sides of the elongate body 12. The first pair of steering wires 36A’, 36B’ are aligned on a first plane 52 passing through a longitudinal axis of the elongate body 12. The second pair of steering wires 38A’, 38B’ of the second steering member 38’ are arranged at radial locations on opposite sides of the elongate body 12 aligned with second plane 54 passing through the longitudinal axis of the elongate body 12. The first plane 52 is orthogonal to the second plane 54.

In another embodiment, the first pair of steering wires 36A’, 36B’ of the first steering member 36’ extend along a first radial location on the elongate body 12 and the second pair of steering wires 38A’, 38B’ of the second steering member 38’ extend along a second radial location on the elongate body 12, and the first radial location is offset from the second radial location by 10-80 degrees. This can be seen clearly in FIG. 7B. In FIG. 7B, steering wire 36A’ is aligned with the first plane 52 but steering wire 36B’ is not aligned with the first plane 52. Rather, steering wire 36B’ is aligned at a radial location that forms an acute angle β with respect to the second plane 54. Acute angle β is in the range of 10-80 degrees.

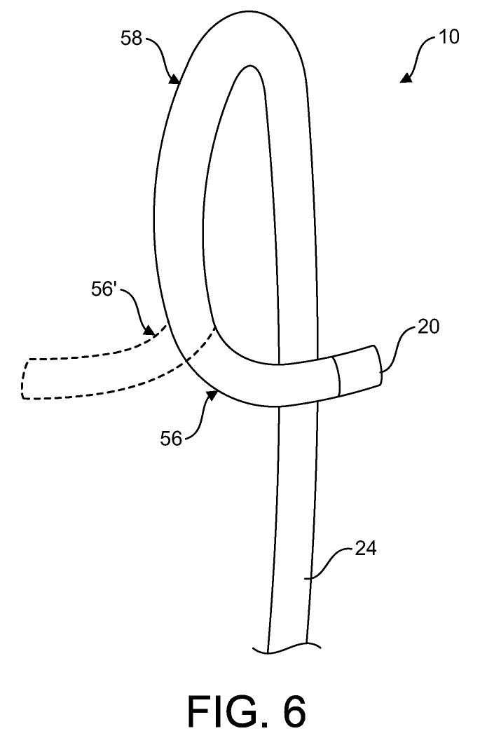

FIG. 6 illustrates a multi-planar shape of the steerable medical device 10 achieved by the arrangement of FIG. 5A, in accordance with embodiments of the disclosure. In FIG. 6, tension is applied to steering wire 36A’ of the first steering member 36’ using the first steering mechanism 26. This causes the elongate body 12 to create a first bend 56 in the distal portion 18. The first bend 56 curves in a first deflection direction aligned in a first plane. Tension is also applied to steering wire 38B’ of the second steering member 38’ using the second steering mechanism 28. This results in the formation of second bend 58 in the distal portion 18. The second bend 58 curves in the fourth deflection direction and is aligned with a second plane. The first plane associated with the first bend 56 is not parallel to the second plane associated with the second bend 58. That is, the first plane and the second plane intersect one another. In some embodiments, the first plane and the second plane are orthogonal. In other embodiments, the first plane and the second plane intersect one another, but are not orthogonal. A mirror image of the multi-planar shape of FIG. 6 is created by tensioning steering wire 36B’ of the first steering member 36 and steering wire 38A’ of the second steering member 38. Additionally, tension applied to steering wire 36B’ would create the first bend 56’ in the opposite direction as illustrated in phantom in FIG. 6.

FIG. 7C is a cross-sectional view along line 7-7 of FIG. 5A illustrating an embodiment of a pull ring, in accordance with embodiments of the disclosure. As illustrated in FIG. 7C, the second pull ring 32’ is configured for routing the first pair of steering wires 36A’, 36B’ of the first steering member 36’ underneath. As illustrated in FIG. 7C, the second pair of steering wires 38A’, 38B’ of the second steering member 38’ are integral with or affixed to the second pull ring 32’. The first pair of steering wires 36A’, 36B’ pass through the interior of the second pull ring 32’ before reaching the first pull ring 30’.

FIGS. 7D-7F are cross-sectional views along line 7-7 of FIG. 5A illustrating embodiments of a pull ring having a non-circular cross-section for routing the first pair of steering wires 36A’, 36B’ of the first steering member 36’. In FIG. 7D, the second pull ring 32’ has an oval or elliptical cross-section. As illustrated in FIG. 7D, the second pair of steering wires 38A’, 38B’ of the second steering member 38’ are integral with or affixed to the second pull ring 32’. The first pair of steering wires 36A’, 36B’ of the first steering member 36’ pass through the interior of the second pull ring 32’ before reaching the first pull ring 30. In FIG. 7E, the second pull ring 32’ includes a pair of diametrically opposed channels 60 configured to guide the first pair of steering wires 36A’, 36B’ underneath the pull ring 32’. In FIG. 7F, the pair of diametrically opposed channels 62 configured to guide the first pair of steering wires 36A’, 36B’ are located on an exterior surface of the second pull ring 32’. While illustrated as rectangular, the channels 60, 62 can be configured as other shapes that correspond to the first pair of steering wires 36A’, 36B’, for example, circular, oval, square, or polygonal.

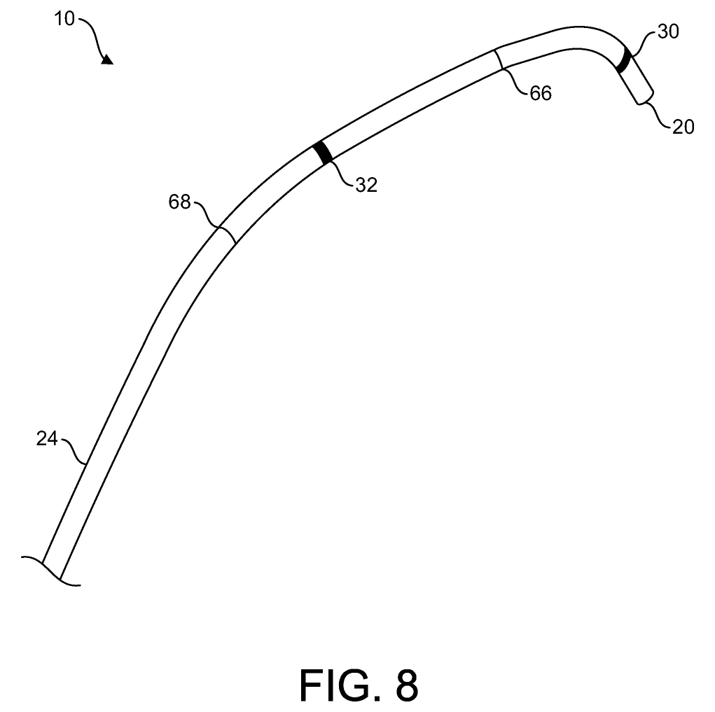

FIG. 8 is a perspective view of a steerable medical device 10, in accordance with embodiments of the disclosure. The elongate body 12 of the steerable medical device 10 of FIG. 8 includes portions of different stiffness. By varying stiffness along the elongate body 12, portions of the elongate body 12 can be configured to bend more or less in response to tension being applied to one or more steering member. Additionally, a proximal section of the device 10 can be articulated by applying tension to the second steering member 38 independently of the distal section because of varying stiffness between the proximal section and the distal section. In FIG. 8, the elongate body 12 has a first stiffness extending from the distal end 18 to a fist joint 66 located proximal to the first pull ring 30. The elongate body 12 has a second stiffness extending from the second pull ring 32 to a second joint 68 proximal of the second pull ring 32. The elongate body 12 has a third stiffness extending from the first joint 66 to the second pull ring 32, and from the second joint 68 to the proximal end 66. In this arrangement, the first stiffness is the softest and is formed of a material having a durometer in the range of 25D - 40D. The third stiffness is the firmest and is formed of a material having a durometer in the range of 63D – 72D. The second stiffness falls between the first stiffness and the third stiffness and is formed of a material having a range of 40D – 63D. While different materials can be used for different portions of the elongate body 12, some embodiments may include varying wall thickness along a length of the elongate body 12 to achieve desired stiffness.

While the present disclosure is directed to steering wires anchored to pull rings within the elongate body, other embodiments can replace pull rings with other anchoring elements. For example, in some embodiments a disk can be used to anchor a distal end of a steering wire. Additionally, in some embodiments, a steering wire can be welded or attached to a support member without using a disc or pull ring. Also, in some embodiments, the steering members are contained withing a guide tube or channel. And in other embodiments, the steering members are wrapped around the elongate body.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A steerable medical device, the device comprising:
an elongate body having a proximal portion including a proximal end and a distal portion including a distal end, and a lumen extending from the proximal end to the distal end;
a first steering member having a proximal end connected to a first steering mechanism and a distal end anchored at a first anchor point in the distal portion of the elongate body; and
a second steering member having a proximal end connected to a second steering mechanism and a distal end anchored at a second anchor point proximal of the first anchor point;
whereby selective actuation of the first steering mechanism and the second steering mechanism allows for the formation of simple, compound, and multi-planar curves of the elongate body.

2. The device of claim 1, further comprising a first pull ring at the first anchor point, wherein the first steering member is attached to the first pull ring.

3. The device of claim 2, further comprising a second pull ring at the second anchor point, wherein the second steering member is attached to the second pull ring.

4. The device of claim 3, further comprising a third steering member having a proximal end connected to a third steering mechanism and a distal end anchored at a third anchor point proximal of the second anchor point.

5. The device of claim 4, further comprising a third pull ring at the third anchor point, wherein the third steering member is attached to the third pull ring.

6. The device of claim 1, wherein the first steering member comprises a first pair of steering wires.

7. The device of claim 1, wherein the second steering member comprises a second pair of steering wires.

8. The device of claim 1, wherein the first steering member and the second steering member extend along a common radial location on the elongate body.

9. The device of claim 1, wherein the first steering member extends along a first radial location on the elongate body and the second steering member extends along a second radial location on the elongate body.

10. The device of claim 9, wherein the first radial location is offset from the second radial location by 90 degrees.

11. The device of claim 9, wherein the first radial location is offset from the second radial location by 10-80 degrees.

12. The device of claim 5, wherein at least one of the second pull ring and the third pull ring is configured for routing the first steering member underneath.

13. The device of claim 5, wherein at least one of the second pull ring and the third pull ring has a non-circular cross-section.

14. The device of claim 5, wherein at least one of the second pull ring and the third pull ring includes a channel for routing the first steering member.

15. The device of claim 1, wherein the elongate body has portions of different stiffness.

16. A steerable medical device, the device comprising:
an elongate body having a proximal portion including a proximal end and a distal portion including a distal end;
a first steering member having a proximal end connected to a first steering mechanism and a distal end anchored to a first pull ring at a first anchor point in the distal portion of the elongate body; and
a second steering member having a proximal end connected to a second steering mechanism and a distal end anchored to a second pull ring at a second anchor point proximal of the first anchor point, the second pull ring being configured for routing the first steering member underneath;
whereby selective actuation of the first steering mechanism and the second steering mechanism allows for the formation of simple, compound, and multi-planar curves of the elongate body.

17. The device of claim 16, wherein the second pull ring has a non-circular cross-section.

18. The device of claim 16, wherein the second pull ring includes a channel for routing the first steering member.

19. A steerable medical device, the device comprising:
an elongate body having a proximal portion including a proximal end, a distal portion including a distal end, and an intermediate portion extending between the proximal portion and the distal portion;
a first steering member having a proximal end connected to a first steering mechanism and a distal end anchored to a first pull ring at a first anchor point in the distal portion of the elongate body;
a second steering member having a proximal end connected to a second steering mechanism and a distal end anchored to a second pull ring at a second anchor point proximal of the first anchor point;
wherein the distal portion has a first stiffness, the intermediate portion has a second stiffness, and the proximal portion has a third stiffness, the first stiffness being less than the second stiffness and the third stiffness;
whereby selective actuation of the first steering mechanism and the second steering mechanism allows for the formation of simple, compound, and multi-planar curves of the elongate body.

20. The device of claim 19, wherein the distal portion extends from the distal end to the second anchor point.
